(19) United States
(12) Patent Application Publication
(10) Pub. No.: US 2017/0339255 A1
(43) Pub. Date: Nov. 23, 2017

(54) DYNAMIC FEEDBACK FOR WEARABLE DEVICES

(71) Applicant: KONINKLIJKE PHILIPS N.V., EINDHOVEN (NL)

(72) Inventors: John Cronin, Bonita Springs, FL (US); Christopher Michael Huffines, Burlington, VT (US)

(21) Appl. No.: 15/532,619

(22) PCT Filed: Dec. 3, 2015
(86) PCT No.: PCT/EP2015/078581

§ 371 Date: Jun. 2, 2017

Related U.S. Application Data
(60) Provisional application No. 62/087,630, filed on Dec. 4, 2014.

(30) Foreign Application Priority Data
May 26, 2015 (EP) 15169231.6

(51) Int. Cl. H04L 29/06; G06F 1/16; A61B 5/00; H04L 29/08

(52) U.S. Cl. CPC H04L 67/42; H04L 67/141; G06F 1/163; A61B 5/0022; A61B 5/742; A61B 5/747

(57) ABSTRACT

Various embodiments disclosed herein relate to wearable devices for capturing biometric, motion, and other wearer measurements and subsequently providing feedback to the user. Various methods and systems described herein include one or more of the following: storing, in a memory, a record specifying applicability criteria, a resource identifier, and a server identifier; obtaining, by a processor in communication with the memory, a user metric captured by the wearable device; comparing the user metric to the applicability criteria to determine that the record is currently applicable; transmitting the resource identifier to the server associated with the server identifier; receiving, in response to transmitting the resource identifier, data for display to a user wearing the wearable device; and effecting display of the data to the user.

### CROSS-REFERENCE TO RELATED APPLICATIONS

The present application claims the priority benefit of U.S. provisional application No. 62/087,630 filed Dec. 4, 2014 and entitled “Health Content Links Based Upon Wearable,” the entire disclosure of which is incorporated herein by reference for all purposes.

### TECHNICAL FIELD

Various embodiments described herein relate generally to wearable technology. More specifically, but not exclusively, various embodiments relate to providing dynamic feedback to a user based on biometric, motion, and other measurements obtained by a wearable device.

### BACKGROUND

Wearable technology may include any type of mobile electronic device that may be worn on the body, attached to or embedded in clothes and accessories of an individual and currently exist in the consumer marketplace. Processors and sensors associated with the wearable technology may display, process or gather information. Such wearable technology has been used in a variety of areas, including monitoring health data of the user as well as other types of data and statistics. These types of devices may be readily available to the public and may be easily purchased by consumers. Examples of some wearable technology in the health arena include FITBIT, NIKE+ FUELBAND, and APPLE WATCH devices.

### SUMMARY

In various embodiments, wearable technology may be linked to one or more websites. For example, a wearable device may be programmed to include access to the website corresponding to the manufacturer of the wearable device (e.g., the NIKE+ FUELBAND device may be linked to the NIKE brand site). This connection between the wearable technology and the branded website may provide a user with additional content and/or help specific to the wearable technology. This additional content may be directly provided by the manufacturer or a third party who is informed about the particular wearable technology in question.

Various embodiments also allow a wearable device to directly access other types of data from websites or networks on the Internet rather than having the information provided directly to the wearable device. For example, hyperlinks may be used in some embodiments used by the wearable device to obtain information that is stored in the Internet. Given that the Internet includes vast amounts of information on various different topics, accessing the Internet directly may provide a user with more information than a single network or database may have available.

Various embodiments described here includes a method for providing information to wearable devices.

Various embodiments relate to a method including storing information in memory of a wearable device, wherein the information pertains to a plurality of topics associated with one or more servers and includes one or more hyperlinks; detecting sensor data via one or more sensors associated with the wearable device; matching data entries stored in memory based on the sensor data and one or more parameters calculated based on the sensor data, the matched data entries pertaining to information identified as being of interest to a user of the wearable device; retrieving the matched data entries in memory, the data entries including one or more hyperlinks; providing the hyperlinks to one or more servers; receiving, from the server, data related to the hyperlink; and displaying the received data on a display for the user to view.

Various embodiments relate to a wearable device for obtaining information. The wearable device according to such embodiments includes memory to store information for various topics pertaining to a plurality of topics associated with one or more servers and includes one or more hyperlinks; one or more sensors associated with the wearable device, wherein sensors are configured to detect sensor data; a processor associated with the wearable device, the processor being configured for: matching data entries in memory, the matched data entries based on a comparison between the sensor data and one or more parameters calculated based on the sensor data, the matched data entries pertaining to information identified as being of interest to a user of the wearable device, and retrieving the matched data entries from memory, the extracted data entries including one or more hyperlinks; and a communication interface configured to communicate over a wireless communication server, wherein the communication interface is configured for requesting information on matched hyperlinks from one or more servers; and receiving data from the one or more servers, the data related to the hyperlink, and a display screen configured to display the received data for the user to view.

Various embodiments relate to a non-transitory computer-readable storage medium, having embodied thereon a program executable by a processor to perform a method for providing information to wearable technology. The method according to such embodiments includes storing information in memory of a wearable device, wherein the information pertains to a plurality of topics associated with one or more servers and includes one or more hyperlinks; detecting sensor data via one or more sensors associated with the wearable device; matching data entries stored in memory based on the sensor data and one or more parameters calculated based on the sensor data, the matched data entries pertaining to information identified as being of interest to a user of the wearable device; retrieving the matched data entries in memory, the data entries including one or more hyperlinks; providing the hyperlinks to one or more servers; receiving, from the server, data related to the hyperlink; and displaying the received data on a display for the user to view.

Various embodiments are described wherein the sensor data corresponds to biometric measurements of the user.

Various embodiments are described wherein the sensor data corresponds to measurements of surroundings of the user.

Various embodiments additionally include providing a designated medical professional with information from the hyperlink in situations where the data associated with the hyperlink corresponds to a medical emergency.

Various embodiments additionally include receiving a message from the medical professional; and sending additional sensor data to the medical professional in response to the message, the additional sensor data relating to a health condition of the user.

Various embodiments are described wherein the parameters are calculated by the wearable device or received from an associated user device communicatively connected with the wearable device over a wireless communication network.

Various embodiments additionally include requesting updated information from one or more servers; and storing information in memory of the wearable device.

Various embodiments relate to a method of obtaining information based on a wearable device, the method including: storing, in a memory, a record specifying applicability criteria, a resource identifier, and a server identifier; obtaining, by a processor in communication with the memory, a user metric captured by the wearable device; comparing the user metric to the applicability criteria to determine that the record is currently applicable; transmitting the resource identifier to the server associated with the server identifier; receiving, in response to transmitting the resource identifier, data for display to a user wearing the wearable device; and effecting display of the data to the user.

Various embodiments relate to wearable device for obtaining information, the device including: a sensor configured to capture sensed data associated with a user wearing the wearable device; a display device; a communication interface; a memory configured to store a record specifying applicability criteria, a resource identifier, and a server identifier; and a processor configured to: generate computed data based on the sensed data, compare a user metric to the applicability criteria to determine whether the record is currently applicable, wherein the user metric includes at least one of the sensed data and the computed data, based on a determination that the record is currently applicable, transmit the resource identifier to the server associated with the server identifier via the communication interface, receive, in response to transmitting the resource identifier and via the communication interface, response data for display to a user wearing the wearable device, and control the display device to output a message to the user based on the response data.

Various embodiments relate to a non-transitory machine-readable medium encoded with instructions for obtaining information based on a wearable device, the non-transitory machine-readable medium including: instructions for storing, in a memory, a record specifying applicability criteria, a resource identifier, and a server identifier; instructions for obtaining, by a processor in communication with the memory, a user metric captured by the wearable device; instructions for comparing the user metric to the applicability criteria to determine that the record is currently applicable; instructions for transmitting the resource identifier to the server associated with the server identifier; instructions for receiving, in response to transmitting the resource identifier, data for display to a user wearing the wearable device; and instructions for effecting display of the data to the user.

Various embodiments are described wherein the memory and processor are components of a user device is communication with the wearable device, obtaining the user metric includes receiving the user metric from the wearable device, and effecting display of the data to the user includes displaying the data via a display device of the user device.

Various embodiments are described wherein the memory and processor are components of a user device is communication with the wearable device, and effecting display of the data to the user includes transmitting the data to the wearable device.

Various embodiments are described wherein the memory and processor are components of the wearable device, and obtaining the user metric captured by the wearable device includes reading the user metric from the memory, the method further including: receiving, at the processor, first data from a sensor of the wearable device, computing, by the processor second data based on the first data, wherein the user metric includes at least one of the first data and the second data, and storing the user metric in the memory.

Various embodiments are described wherein effecting display of the data to the user includes displaying the data via a display device of the wearable device.

Various embodiments are described wherein the user metric is indicative of a medical emergency, the method further including: after transmitting the resource identifier, receiving a message from a medical professional device associated with a medical professional; and transmitting an additional user metric to the medical professional device, wherein the additional user metric includes at least one of sensor data and computed data.

Various embodiments are described wherein the user metric is indicative of a medical emergency, the method further including, after transmitting the resource identifier, establishing a two-way communication session between the processor and a medical professional device associated with a medical professional.

Various embodiments additionally include receiving, from at least one of the server associated with the server identifier and an additional source server, at least one additional record specifying additional applicability criteria, an additional resource identifier, and at least one of: the server identified by the record, the additional source server, and an additional destination server; and storing the additional record in the memory for future comparison to user metrics.

### BRIEF DESCRIPTION OF THE DRAWINGS

In order to better understand various example embodiments, reference is made to the accompanying drawings, wherein:

FIG. 1 illustrates an example of a system for providing hyperlinks to a wearable device as described herein according to an embodiment;

FIG. 2 illustrates an example of a wearable device as described herein according to an embodiment;

FIG. 3 illustrates an example of a reference link database as described herein according to an embodiment;

FIG. 4 illustrates an example of a method for the link program as described herein according to an embodiment;

FIG. 5 illustrates an example of a method for the reference software module stored in the wearable device and/or user device according to an embodiment;

FIG. 6 illustrates an example of a computing device architecture that may be utilized to implement the various features and processes described herein according to an embodiment;

FIG. 7 illustrates an example of a method whereby the reference software module determines related hyperlinks as described herein according to an embodiment;

FIG. 8 illustrates an example of a display on the wearable device as described herein according to an embodiment;

FIG. 9 illustrates swim lanes diagram among six elements (e.g., display, sensors, base algorithm module, reference link database, reference software module, network) of the example system described herein according to an embodiment;

FIG. 10 illustrates an example of a swim lane diagram among elements (e.g., display, sensors, base algorithm module, reference link database, reference software module, network, and some third party) of the example system as described herein according to an embodiment; and

FIG. 11 illustrates an example of a viewers with reference to a medical emergency situation using the medical emergency network according to an embodiment.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., or), unless otherwise indicated (e.g., “or else” or “or in the alternative”). Additionally, the various embodiments described herein are not necessarily mutually exclusive and may be combined to produce additional embodiments that incorporate the principles described herein.

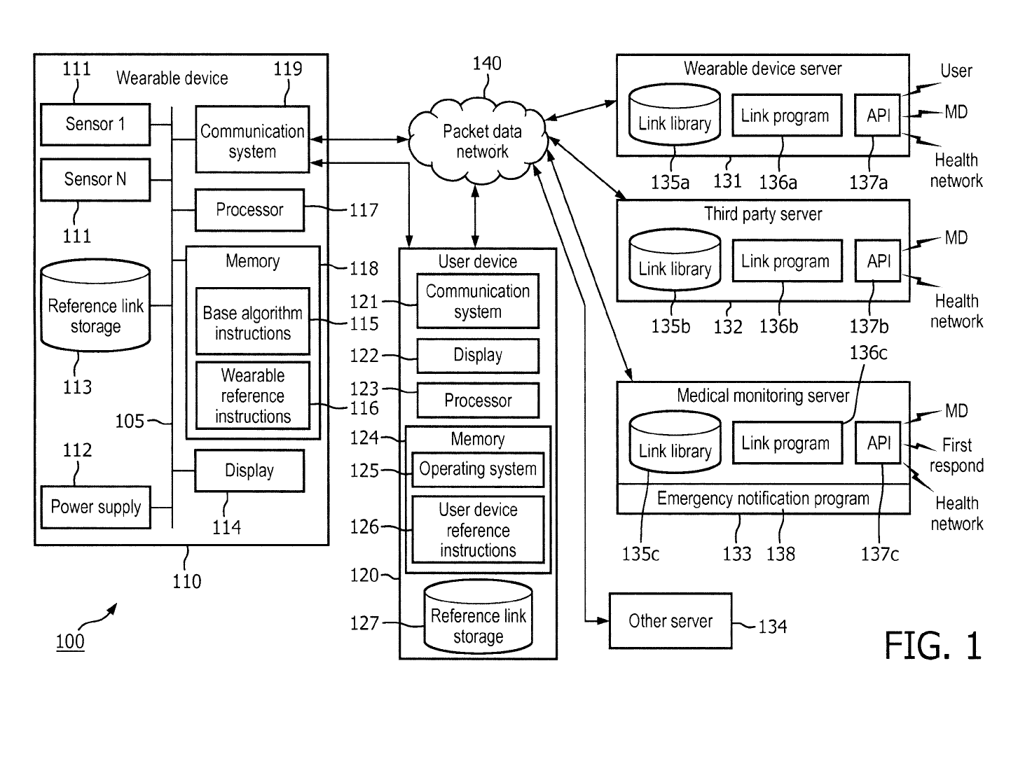

Various embodiments described herein achieve various functionality through the execution of instructions by a processor. It will be understood that, while various examples are described in the context of instructions actively performing steps or other actions, any such actions will actually be performed by the processor that executes such instructions. FIG. 1 illustrates an example of a system 100 for providing hyperlinks to a wearable device as described herein. Overall, the system 100 may include a number of elements such as a wearable device 110, a user mobile device 120, and a plurality of networks 130. The elements in the system may be connected to each other through the use of a packet data network 140.

The packet data network 140 may include, for example, a carrier network, a local area network (LAN), or a wide area network (WAN) such as the Internet. As such, the packet data network 140 may provide access to various servers (not shown) which provide access to services or resources. Such services or resources may be identified by a uniform resource identifier (URI); as such, various hyperlinks described herein may include a specification of one or more URIs. Following a hyperlink may entail transmitting a request to a server associated with a URI and receiving a response therefrom. It will be apparent that various servers may be provisioned as virtual machines within a cloud computing environment. Various references to “the cloud” used herein will be understood to refer to various services or resources provided to external users from within such a cloud computing environment.

The wearable device 110 may be a wearable device that currently exists in today's market (e.g., NIKE+ FUELBAND, FUELBAND, APPLE WATCH wearable devices) or a wearable device that has yet to be developed on introduced to the market. The wearable device 110 may be worn or carried by a user. Generally, the wearable device 110 may include a one or more sensors 111, a power supply 112, a reference link storage 113, a display 114, a processor 117, a memory 118, and a communication system 119. As shown, the memory 118 may store a base algorithm instructions 115 and reference instructions 116 for execution by the processor 117. It will be understood that these instructions may be alternatively or additionally stored in a non-volatile storage device such as the storage device storing the reference link database or another storage device (not shown). For example, the instructions may be stored in a flash memory or an electronic read only memory (ROM) until they are to be executed by the processor, at which point they are copied to the memory 118. As used herein, the term storage will be understood to refer to non-volatile memories.

The one or more sensors 111 may include any type of sensor that is known in the art. Generally, the sensors 111 may be used, for example, to detect and obtain sensor data (e.g., biometrics) about the user (e.g., heart rate, blood pressure) or obtain sensor data regarding the surrounding environment (e.g., temperature, humidity). Sensors may also be used for other purposes such as a step counter (e.g., pedometer). The sensors 111 may be mounted on the wearable device 110 or may be external devices that are separately wearable by the user and communicate with the wearable device 110 wirelessly or via a wired connection.

The power supply 112 may be used to provide power used by the wearable device 110 for maintaining operation of the overall device. In various embodiments, the power supply 112 may include a battery, one or more capacitors, a powered USB interface, or a power cord and plug. In some embodiments, the power supply may be chargeable using an external power source (e.g., battery charger).

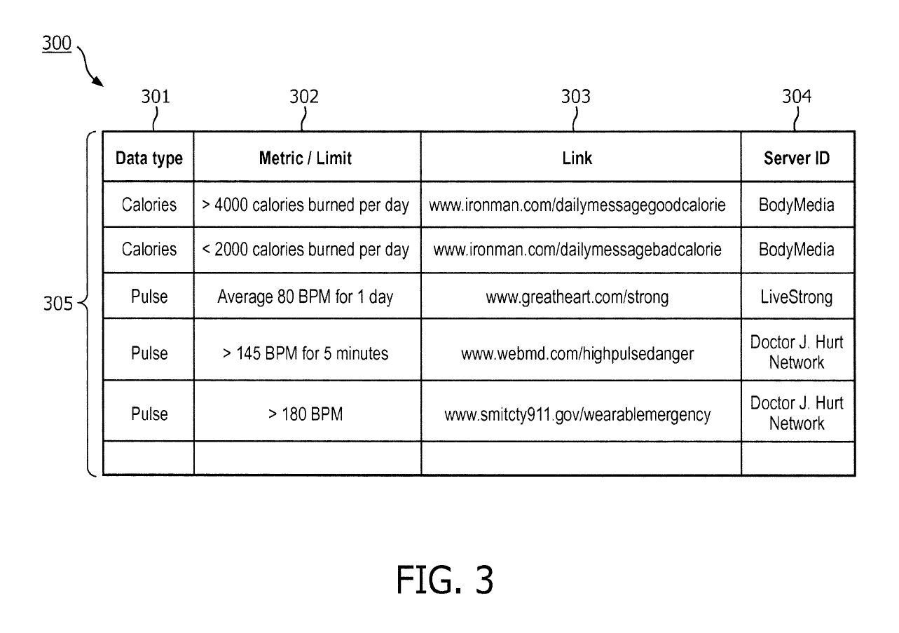

The reference link storage 113 may be a storage device such as read-only memory (ROM), random-access memory (RAM), magnetic disk storage media, optical storage media, flash-memory devices, or similar storage media. The reference link storage 113 may store a database including one or more hyperlinks (also referred to as reference links). These hyperlinks stored in the reference link storage 113 may be hyperlinks that the wearable device 110 may process to obtain information from the packet data network 140. For example, where the hyperlink specifies a URI, the wearable device 110 may transmit an HTTP GET request via the packet data network 140 to a server associated with the URI. As another example, in some embodiments, rather than accessing the server identified by the URI directly, the wearable device may transmit a request identifying the URI to one of the networks 131, 132, 133, 134 such as, for example, a network identified by an entry in the reference link storage 113, an example of which will be described in greater detail below with respect to FIG. 3.

In some embodiments, some hyperlinks stored in the reference link storage 113 may have been originally stored in the wearable device 110 during initial manufacture of the wearable device 110. In some embodiments, hyperlinks may be provided to the wearable device 110 by one or more third parties and subsequently stored in the reference link storage 113. In some embodiments, the user may manually input hyperlinks into the wearable device 110 through the use of a user input device (not shown) (e.g., one or more buttons, a keypad, or a wireless or wired connection to another device such as a mobile device or personal computer) and these hyperlinks may also be saved in the reference link storage 113. More information about how the database stored in the reference link storage 113 operates will be provided below with respect to FIG. 3.

Generally, the display 114 provides the user with a view of information on the wearable device 110. The information may include hyperlinks, sensor data, or corresponding data obtained from the hyperlinks from the internet. The display 114 in the wearable device 110 may be virtually any device for communicating information to a user such as, for example, a segment display or a video display, and may be implemented using various technologies such as, for example, light emitting diode (LED) display, organic LED display, liquid crystal display (LCD), or thin-film transistor (TFT) display. In some embodiments, the display 114 may also be a touchscreen display. A touchscreen display may allow the user to provide input into the wearable device 110 whereby the user may be able to select, input, and modify information displayed thereon. It will be apparent that, in various embodiments, additional or alternative input and output devices that are not shown may also be provided. For example, the device 110 may include one or more LED status lights, a speaker, or a haptic feedback engine for output to the user and one or more buttons, a keypad (physical or soft via a touchscreen), a microphone, or a camera for user input.

The base algorithm instructions 115 of the wearable device 110 may be used by the processor 117 to conduct the various processes and calculations for the wearable device 110. The specific implementation of the base algorithm instructions will be highly dependent on the overall goal or purpose of the wearable device 110; for example, a wearable device for tracking a heart rate will include different base algorithm instructions 115 from a wearable device for tracking steps taken. For example, the base algorithm instructions 115 may be used to calculate one or more parameters based on measured sensor data obtained from the plurality of sensors 111. For example, in some embodiments wherein the sensors 111 include a step counter, and the base algorithm instructions 115 may be used to take the number of steps taken by the user and calculate possible parameters such as distance traveled by the user or number of calories burned by the user.

The reference instructions 116 in the wearable device 110 may be used by the processor 117 to provide one or more related hyperlinks for the user to view based on obtained sensor data. For example, the reference instructions 116 may compare the parameters calculated by the base algorithm instructions 115 and perform a look-up in the reference link storage 113 for any related information relevant to the calculated parameters. Following with the earlier example of a step counter, if the base algorithm instructions 115 provides, as an output, the number of calories burned, the reference instructions 116 may then look for entries within the reference link storage 113 pertaining to the provided calorie amount. If the reference instructions 116 find a relative match between the calculated parameter from the base algorithm instructions 115 and an entry in the reference link storage 113, the reference software instructions 116 may provide the relative match to the user to view on the display 114.

In various embodiments, the reference instructions 116 may, upon finding a relative match, obtain information from a network 130 through the packet data network 140 by accessing the hyperlink obtained by, for example, transmitting an HTTP GET request to the server identified by a URI specified by the hyperlink. Once the information is accessed from the packet data network 140, the reference instructions 116 may provide the information to the user on the display 114 of the wearable device 110.

The wearable device 110 may include one or more processors 117. The processor 117 may be virtually any device capable of performing the functions described herein including the functions described above in connection with the base algorithm instructions 115 and the reference instructions 116. For example, the processor 117 may include one or more microprocessors, one or more field-programmable gate arrays (FPGA), or one or more application-specific integrated circuits (ASIC). In some embodiments, the processor may not utilize stored instructions to perform some or all of the functions described herein; for example, an ASIC may be hardwired to perform one or more of the functions describe above with reference to the base algorithm instructions 115 and the reference instructions 116. In some such embodiments, the base algorithm instructions 115 or the reference instructions 116 may be omitted because they are already embodied in the processor 117 without the need for stored instructions.

The processors 117 may be in communication with one or more memory devices 118 that may be used for storing instructions or data on the wearable device 110. The memory devices 118 may include various memories such as, for example L1, L2, or L3 cache or system memory. As such, the memory 430 may include static random access memory (SRAM), dynamic RAM (DRAM), flash memory, read only memory (ROM), or other similar memory devices. Data that may be stored in memory 118 may include the sensor data from the plurality of sensors 111, parameters calculated by the base algorithm instructions 115, or information obtained from a network after accessing a hyperlink.

As will be understood, devices referred to herein as “storage” or “memory” may both be considered to be “non-transitory machine-readable media.” As used herein, the term “non-transitory” will be understood to exclude transitory signals but to include all forms of information storage, including both volatile and non-volatile memories.

The wearable device 110 may further include a communication system 119 used to facilitate communication (e.g., wireless or wire line communication) between the wearable device 110 and other elements in the system (e.g., the user device 120 and networks 130). The communication system 119 may be implemented using various technologies, including 3G, 4G, IEEE 802.11 Wi-Fi, near field communication (NFC), or Bluetooth.

In various embodiments, most, if not all, the elements described above with respect to the wearable device 110 may be connected to a single bus 105. The bus 105 facilitates communication between each of the elements of the wearable device 110. Even though FIG. 1 illustrates an embodiment where a single bus 105 is used, in other embodiments, two or more connections may be provided between elements of the wearable device 110 to facilitate communication among the elements. In other embodiments, the single bus 105 may be replaced by multiple buses arranged in, e.g., a hierarchy. For example, a system bus may connect the reference link storage 113, processor 117, memory 118, communication system 119, and a peripheral bus bridge (not shown), while a second peripheral bus may connect the peripheral bus bridge with the sensors 111 and display 114. Various additional arrangements for two or more buses will be apparent.

As shown in the example of FIG. 1, the system 100 may also include a user device 120. User device 120 may include, for example, a smartphone, tablet, personal computer, or any other device that may provide additional computing functionalities to the wearable device 110. The user device 120 may be in periodic or continuous communication with the wearable device 110 through the use of the communication system 119 of the wearable device 110 and a communication system 121 of the user device 120. For example, the wearable device 110 may establish communication with the user device 120 whenever the user tethers the wearable device 110 to the user device 120 via a cable (e.g., a USB cable), whenever the wearable device 110 joins a network (e.g., a wireless LAN) to which the user device is attached, or whenever the wearable device 110 comes within NFC or Bluetooth range of the user device 120 for direct communication. In some embodiments, continuous communication may be established via the packet data network 140 even when the wearable device 110 is geographically remote from the user device 120; for example, the wearable device 110 may communicate with the user device 120 via a 4G LTE carrier network, the Internet, and an Internet service provider (ISP) network (all of which may be represented as the packet data network 140).

The user device 120 may be any device such as, for example, a mobile phone, tablet, personal computer, game console, or television set top box. In various embodiments, the user device 120 includes a communication system 121, a display 122, a processor 123, a memory device 124, and a reference link storage device 127. Each of these components 121, 122, 124, 127 may be implemented by various hardware devices such as those described above with respect to the similarly named devices 119, 114, 117, 118, and 113 of the wearable device 110 . . . . As shown, the memory device 124 stores various instructions 125, 126 for execution by the processor 123. As with the instructions 115, 116 of the wearable device 110, it will be understood that the instructions 125, 126 may additionally or alternatively be stored in a non-volatile storage device such as, for example, the same devices as the reference link storage 127. The operating system 125 may coordinate the various basic functions of the user device 120. For example, where the user device 120 is a mobile phone or tablet, the operating system 125 may be the APPLE IOS or GOOGLE ANDROID operating system.

The user device reference instructions 126 include instructions that are similar to or complement the wearable reference instructions 116. For example, in some embodiments, the wearable reference instructions 116 may simply pass sensed data from the sensors 111 to the user device 120 and receive links for display in return. In such embodiments, the user device reference instructions may calculate parameters from the received data, determine which links within the reference link storage 127 are applicable to the received or calculated data, and return the link to the wearable device. In other embodiments, the two sets of reference instructions 116, 127 may operate in parallel; for example, some links may be locally stored by the wearable device 110 while others may be stored in the reference link storage. In some such embodiments, simple link entries that only use data immediately available to the wearable device 110 (e.g., sensor data and some calculated parameters) may be stored in the reference link storage 113 while more complex link entries may be stored in the reference link storage 127 at the user device 120. For example, the user device 120 may store those link entries that use data that is to be retrieved from other sources or that requires parameters that are to be computed by the user device 120 to determine link applicability. Various additional divisions or duplications of responsibility between the wearable device 110 and user device 120 will be apparent. In a similar manner, these and other divisions or duplications of responsibility between the wearable device 110 or user device 120 and one or more virtual machines deployed in a cloud computing data center (not shown) will be apparent.

The system 100 may also include a one or more additional servers 131, 132, 133, 134. The servers 131, 132, 133, 134 may be accessed by the wearable device 110 or the user device 120 through the use of their respective communication systems 119, 121. Access may be established through the use of the packet data network 140. Some servers that may be included in the plurality of servers may be, for example, a wearable device server 131, third party servers 132, medical monitoring servers 133, or any other server 134 accessible via the packet data network 140. IT will be understood that, while each server 131, 132, 133, 134 is referred to in the singular form, that the functionality described in connection with each server 131, 132, 133, 134 may be divided among multiple servers, or a network of servers. Furthermore, each server 131, 132, 133, 134 may be implemented as one or more virtual machines (VM) resident on one or more physical servers among one or more cloud computing data centers. For example, each of the link library storage 135a, link program 136, and API 137a may be implemented within three respective virtual machines which may be co-resident in a single data center or geographically distributed among multiple data centers.

Each of the example servers 131, 132, 133, 134 may include a link library 135a,b,c, a link program 136a,b,c, and an application programming interface (API) 137a,b,c. The link library 135a,b,c may store various hyperlinks related to the subject matter being covered by the particular server. The link library 135a,b,c may be used to populate the reference link storage 113, 125 in the wearable device 110 or user device 120, respectively. For example, if a server 132 relates to a third party health server concerned with health care, the link library 135b may include hyperlinks for information accessible from the server 132 or another server accessible via the packet data network 140 regarding various health-related issues and solutions for health-related issues. The link libraries 135a,b,c may be used by the plurality of servers 131, 132, 133 to retrieve relevant information from the packet data network 140 and provide to the user upon request. Thus, the user may access the information from servers (not shown) via the packet data network 140 using the hyperlinks, as well as save the information into memory for access at some future time.

The link program 136a,b,c determines which hyperlinks are related or which hyperlinks may be of interest to a particular user. The link program 136a,b,c may receive sensor data from the wearable device 110 or calculated parameters from the reference software instructions 116, 123. The link program 136a,b,c may then review the entries available in the link library 135a,b,c and figure out what entries to send back to the user. As described above, the reference software module 116, 123 of the wearable device 110 or the user device 120 may provide the results from the base algorithm instructions 115 (e.g. a parameter such as a number of calories burned) to one or more of the servers 131, 132, 133, 134. Any resulting matches that the server 130 may find in the link library 135a,b,c may then be sent to the wearable device 110 and/or user device 120.

The link program 136a,b,c may also retrieve information from one or more hyperlinks stored in link library 135a,b,c. The information may then be stored in the server or provided to the wearable device 110 or user device 120. The information may then displayed on the display 114, 124 of the wearable device 110 or the user device 120 for the user to view.

The API 137 found in some of the servers 131, 132, 133 may be used, for example, by the user or third parties to populate information into the link library 135a,b,c. In some embodiments, doctors (MD) or additional third party servers may be able to provide relevant information or hyperlinks that may be stored in in the link library 135a,b,c. In this way the link library 135a,b,c may be maintained and kept up-to-date through the input of one or more outside parties (e.g., doctors, health servers).

In some embodiments, link entries to be pushed to a user device 120 or wearable device 110 may be dynamically created based on data reported by the user device 120 or wearable device 110. For example, in some embodiments, the wearable device 110 or user device 120 may periodically (e.g., during database sync) report sensor readings or calculated parameters to the servers 131, 132, 133, 134. Using this information, the link program 136a,b,c or another program via the API 137a,b,c may generate rules specific to the user. For example, an average calorie burn per day may be used to set thresholds for “good” and “bad” messages (e.g., the first tow entries described below with respect to FIG. 3). A user that more consistently reaches high levels of calorie burn may be provided an entry that does not trigger an encouragement message until an exceptional calorie burn relative to that user has been achieved (e.g., 6000 calories instead of 4000 calories). As another example, similar thresholds may be tailored to data reported about the user such as age, gender, weight, and height.

It should be noted that the servers may also include other programs and modules that may be stored and run on servers accessible via the packet data network. For example, as seen in FIG. 1, the medical monitoring server 133 may include an additional module (e.g., emergency notification program 138). These modules may provide further functionalities not covered with the link library 135a,b,c, link program 136a,b,c or the API 137a,b,c. In various embodiments, the servers 131, 132, 133, 134 may include any number of modules or programs outside of the ones described above that may further be used to facilitate each server and its particular functionality or purpose. As will be appreciated, the link programs 136a,b,c, API 137a,b,c, and any other “modules” 138 may be implemented as hardware or hardware in combination with software such as, for example, instructions stored in memory to be executed by a processor (not shown).

As illustrated in FIG. 1, an example of a server may be the wearable device server 131. The wearable device server 131 may be associated with a particular manufacturer or brand of the wearable device in question. For example, if a user has a NIKE+ FUELBAND device, the wearable device 110 may be able to connect to a wearable device server associated with NIKE. The NIKE company server may include information that may be found useful for wearable devices manufactured by NIKE. Such information may be initially stored in the link library 135a and updated accordingly through the use of the API 137a by the manufacturer or other third parties. The link program 136a may obtain information from the user through the wearable device 110 or the user device 121 and then determine what type of information or hyperlinks to provide from the link library 135a back to the user.

Also included in the plurality of servers may be the third-party server 132. Third party servers 132 may include, for example, a server run by the American Heart Association. Generally, third party servers 132 are not associated with a particular manufacturer or brand. Rather, these third party servers 132 may be related to other types of subject matter. For example, as indicated above with the embodiment of the American Heart Association, third party servers 132 may be directed towards providing information to users related to health. For example, the American Heart Association may provide relevant health information based on their area of expertise (e.g. cardiac care). Alternatively, the third-party server may include general health based information. Similar to the operation of the wearable device server 131 described above, the information for the third party-server may be stored in the link library and updated accordingly through the use of the API 137. A determination as to which information a user may be interested in may be performed by the link program 136b.

Another example of a server in the plurality of servers may also include the medical monitoring server 133. The medical monitoring server 133, like the other servers described above, may also be accessible by the wearable device 110 or user device 120. The medical monitoring server 133 may be used to provide long-term monitoring of a health condition of the user. In some embodiments, a user may have heart trouble (e.g., susceptible to a heart attack). This medical monitoring server 133 may be directed at ensuring that the user is doing well and may alert appropriate parties or individuals if the well-being of an individual declines. In particular, the link library 135c and the link program 136c of the medical monitoring server 133 may provide specialized information relevant to medical emergencies that may arise based on sensor data or parameters transmitted from the user using the wearable device 110 or user device 120. The medical monitoring server 133 may also include information related to preventative measures that may be taken by the user based on the same sensor data or parameters transmitted. The specialized information or information related to preventative measures may be provided by third parties using the API 137c associated with the medical monitoring server 133.

As indicated above, the servers may include additional components or programs. This may be seen with the medical monitoring server 133 illustrated in FIG. 1. The medical monitoring server 133, may include, for example, an emergency notification program 138. This program may be provided to alert the user or other medical professionals (e.g., doctors, hospitals) based on the sensor data or parameters transmitted to the medical monitoring server 133. The emergency notification program 138 may include a set of rules used to evaluate one or more sensor data or parameters obtained from the user to determine an overall situation. Based on the outcome of the evaluation, the emergency notification program 138 may identify a user's condition as being an emergency condition. If an emergency condition is identified, the emergency notification program 138 may interface with a variety of contacts (e.g., first responders including police, fire, hospital, emergency room). The emergency notification program 138 may also be provided to mediate communication between the user and the appropriate first responder during the course of the response. For example, information about the user may also be transmitted to better inform the medical professionals about the potential emergency.

The plurality of servers may also include any other server 134 that may be accessible via the packet data network 140. These other servers 134 may relate to other types of subject matter not covered above such as weight loss or fitness. Information stored in these other servers 134 may be helpful for a user during training for a marathon and could then be provided to the user upon request based on the provided sensor data or parameters transmitted to the other server 134.

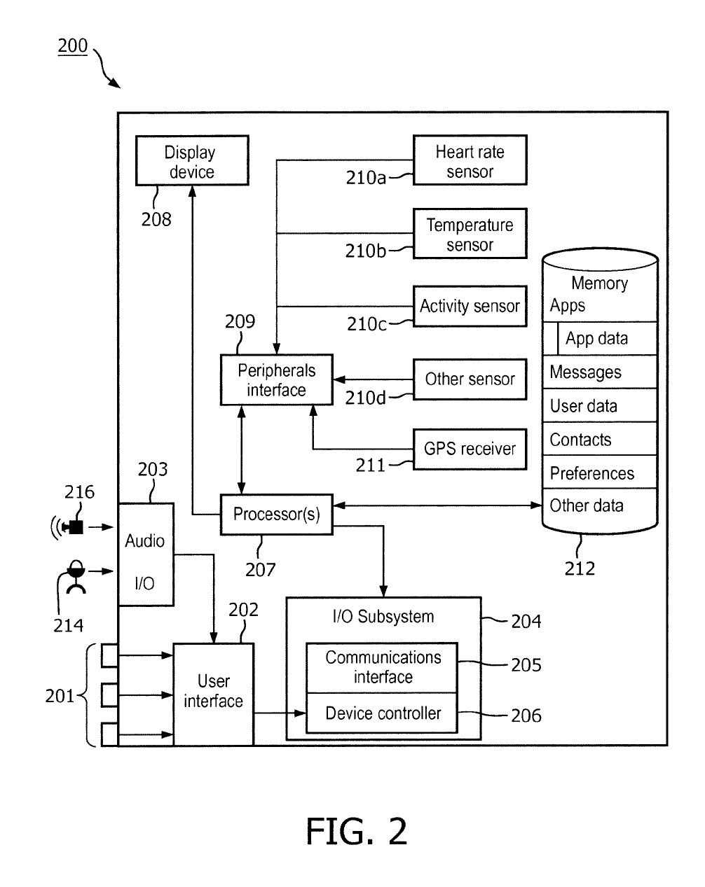

FIG. 2 illustrates an example of a wearable device 200 as described herein. In various embodiments, the wearable device 200 corresponds to the wearable device 110 illustrated in FIG. 1.

The wearable device 200 in FIG. 2 may include a number of different elements. It should be noted that a various alternative wearable devices may include additional elements or omit some of the elements shown in FIG. 2. In this way, FIG. 2 may be considered an example of an embodiment of a wearable device.

The wearable device 200 may include a one or more physical buttons or touch screens 201 operatively connected to a user interface 202 of the wearable device 200. The buttons or touch screens 201 may be provided for the user to interact with the various features of the wearable device 200 through the use of the user interface 202. The user interface 202 could also be used to inform the user of the various features of the wearable device 200 and then obtain user input through the use of the buttons and/or touch screens 201. To inform the user, embodiments may include the user interface 202 having a display (not shown) or the user interface 202 may work in conjunction with the display device 208 where information and features are shown to the user.

In various embodiments, buttons 201 used in the wearable device 200 may be physical buttons that may be pressed or toggled. Other types of buttons 201, such as the crown button in the APPLE WATCH device may also be implemented. The touch screens 201 may be implemented by using capacitive touch capabilities for users to interact with through touch.

The wearable device 200 may also include audio input/output (I/O) elements 203. Such I/O elements 203 may include microphones 214 and speakers 216 and be connected to the user interface. In an embodiment of the present disclosure, the user may be able to utilize the I/O elements 203 to interact with the wearable device 200 and provide user input. For example, the user may instruct the wearable device 200 to perform a task using a voice-recognized instruction. In another embodiment, the I/O elements 203 may be used by the wearable device 200 to inform the user information. For example, information may be provided through an audio output for the user to listen to. In this way, an embodiment of the wearable device 200 may be able to use any combination of buttons, touch screens 201 and audio I/O elements 203 in order to obtain information from the user.

In an embodiment of the present disclosure, the user interface 202 may include a personal digital assistant (PDA) system (not shown). PDA systems may be, for example, similar to systems such as the APPLE SIRI or MICROSOFT CORTANA assistant systems. These PDA systems may be implemented to facilitate interaction between the user and the wearable device 200 (e.g., allow the user to provide user inputs) or to provide a way that the wearable device 200 may provide information to the user. These PDA systems may work in conjunction with the audio I/O elements 203 to carry out the above feature.

As illustrated in FIG. 2, the user interface 202 of the wearable device 200 may be connected to the I/O subsystem 204.

The I/O subsystem 202 may include a communication interface 205 and device controller 206. The communication interface 205 may be used by the wearable device 200 to obtain information from other sources (e.g., user device, servers) through available communication methods (e.g., wireless communications such as Wi-Fi, Blue tooth, or NFC). The device controller 206 may be used to translate user input obtained from the user interface 202 and translate the user input into recognizable instructions that the wearable device 200 may use. For example, the device controller 206 may translate button or touch screen interactions in conjunction with the user interface 202 to determine what the user wishes the wearable device 200 to perform.

The wearable device 200 may also include one or more processors 207 such as, for example one or more microprocessors, one or more field-programmable gate arrays (FPGA), or one or more application-specific integrated circuits (ASIC). The processors 207 may be used by the wearable device 200 to perform various processing within the wearable device 200.

The processors 207 may be connected to a display device 208. The display device 208 may be used to display information for the user of the wearable device 200 to view. Such information displayed may include possible configurable settings for the wearable device 200, sensor data measured by the wearable device 200, and results from the base algorithm (e.g., parameters). The display itself may be any type of display or screen available including retina display, full color screens and liquid crystal displays (LCDs).

The processors 207 may also be connected to a peripherals interface 209. The peripherals interface 209 may be an interface for the plurality of sensors 210 that may be included in the wearable device 200. For example, the peripherals interface 209 may be a bus bridge between a system bus and a peripherals bus.

The wearable device 200 may include a variety of different sensors 210a,b,c,d. The sensors 210a,b,c,d included may be based on a particular functionality of the wearable device 200. The sensors 210a,b,c,d may include, for example, heart rate 210a sensor, temperature sensor 210b, and activity 210c sensor. Additional or alternative types of sensors 210d may be included in the wearable device 200 as designed for a particular wearable device. It should be noted that FIG. 2 is an example of an embodiment and that other embodiments may include more or less sensors.

The wearable device 200 may also include a global positioning system (GPS) receiver 211. The GPS receiver 211 may be used to calculate a user location and relate that information as needed. As described above, an embodiment related to the medical monitoring server may include providing the user location to medical professionals (e.g., doctors, hospitals) if an emergency is detected. The user location may be used by medical professionals to locate the user.

As seen in FIG. 2, the processor 207 may also be connected to memory 212. The memory 212 may store a plurality of different types of information and applications. As shown in the figure, the memory 212 may store one or more applications (e.g., programs that may be run by the processor 207 on the wearable device 200), application data used to run the wearable device 200, sensor data stored by the wearable device 200, messages (e.g., short message service (SMS) messages, multimedia message service (MMS) messages, or raw data), user data identifying the user (e.g., name, ID number, address, phone number and health records), contacts (e.g., phone numbers and/or e-mail addresses of user's friends and acquaintances the user wishes to communicate with) and user preferences. The user preferences may pertain to customizable settings that the user has selected for the operation of the wearable device 200. It should be noted that the listing above and illustrated in FIG. 2 is purely illustrative. The memory 212 of the wearable device 200 may be able to store other type of data not listed above.

FIG. 3 illustrates an example of a reference link database 300 as described herein. It should be noted that the reference link database may be found in the wearable device 110 or the user device 120 of FIG. 1 (e.g., stored in the reference link storage 113, 125).

The reference link database 300, as illustrated in FIG. 3, may be used to store various types of information and, as such, may include a data type field 301, metric/limit field 302, hyperlinks field 303 and server identification field 304. Each set of information may be considered an entry 305 and the reference link database 300 may store a plurality of entries 305.

The data type field 301 may store information identifying a particular entry and its relation to a particular subject matter. For example, some data types may include, for example, calories and pulse. In these cases, the corresponding entries 305 stored in the reference link database may be related to instances where the reference software module is looking for matches based on sensor data or parameters from the base algorithm related to calories or user pulse, respectively. In this way, the data type 301 may be used as a way for the reference instructions to determine which entries are relevant to the sensor data being measured. It should be noted that virtually any data types recognized by the device may be included. FIG. 3 should be viewed as an example embodiment and that other embodiments may include any other possible data type that may be detected or measured by the wearable device or calculated from data detected or measured by the wearable device.

In various embodiments, the data type field 301 may be used to determine which entries 305 should be stored on the wearable device or user device by the link program. For example, in some embodiments, many different types of wearable devices including different types of sensors may implement reference instructions such as those described herein. In such embodiments it may not be desirable to automatically load all link entries available at a server into a wearable device. For example, a device that does not include any sensor for measuring pulse would not be loaded with entries of “pulse” data type. As such, the wearable device may report the relevant data types to the server(s) upon registration or sync or the server may infer the relevant data types from the types of information reported by the wearable device. Thereafter, only those entries associated with a relevant data type will be loaded onto the wearable device or associated user device.

The metric/limit field 302 illustrated in FIG. 3 may store data that provides further classification related to the data type 301 of a particular entry 305. Various embodiments shown in the figure show that the metric/limit field 302 may define a rule used to further detail an entry 305 such as, for example, to be used in determining whether a particular entry 305 is applicable and, consequently, that the corresponding link should be followed. For example, in the first entry shown in FIG. 3, the first entry has a metric/limit rule that specifies “more than 4,000 calories are burned in a day.” In an embodiment, if sensor data is obtained that falls under this classification, the following hyperlink stored in the associated link field 303 of the same entry 305 may then be provided to the user. In this embodiment, the hyperlink “www.ironman.com/dailymessagegoodcalorie” may be provided to the user to view. The hyperlink 303 may provide information that could be beneficial for the user. The hyperlink 303 may also provide a beneficial message (e.g., words of encouragement).

Alongside the hyperlink filed 303, a server identification field 304 may store an identifier (ID) of a server (e.g., a server name, domain name, IP address, or other value for identifying a server) to also be provided to the user. The server identification field 304 may be used to identify a particular server that may contain information that is helpful for the user based on the sensor data. The server identification field 304 may be related to the hyperlink field 303 but may also reference other related information not disclosed in the hyperlink stored in the hyperlink field 303. For example, in some embodiments, the wearable device may not access the link in the hyperlink field 303 directly and, instead, may forward the link identification to the server identified in the server ID field 304. In some such embodiments, rather than storing a URI in the link field 303 as shown, an alternative more compact identifier known to the server identified in the server ID field 304 may be used. For example, in the example of the first entry 305 having been determined to be applicable (i.e., the base algorithm instructions reported over 4000 calories burned in a day), the wearable device or user device may forward the link “www.ironman.com/dailymessagegoodcalorie” (or, in alternative embodiments, a non-URI identifier such as, for example, a unique alphanumeric value) to the BodyMedia server, rather than performing a DNS lookup to determine the server associated with “www.ironman.com” and forwarding a request to that identified server.

It should be noted that other embodiments may include additional or alternative types of data being stored in the reference link database 300. Related messages for a particular entry may be stored so that these messages may be provided to the user, alongside or to replacing hyperlinks. Furthermore, there may be other types of information stored in the reference link database that may be used to further detail and classify entries to provide further specificity to a particular entry so that the user may obtain information that is more likely to be desired by the user. For example, particular biometrics of the user (e.g., age, gender) may be used to further distinguish whether a particular entry would be useful for a user to view.

As another example of an alternative or additional field, the reference database may include a field identifying one or more sensor measurements, calculated parameters, or other information that should be forwarded to the server in the server ID field 304 when an entry is applicable. For example, a field may indicate for the first entry 305 that the current calorie count and an average calorie count (as reported by, e.g., the wearable device or user device) for the last week should also be forwarded to the BodyMedia server such that the BodyMedia server can tailor the response to the user (“you're doing better” vs. “keep it up”). As another example, a field may be included with a message template that is to be filled with data returned from the server. In this way, static content associated with the message to be delivered when a rule is applicable may be preloaded onto the user device or wearable device and supplemented with information returned by the server.

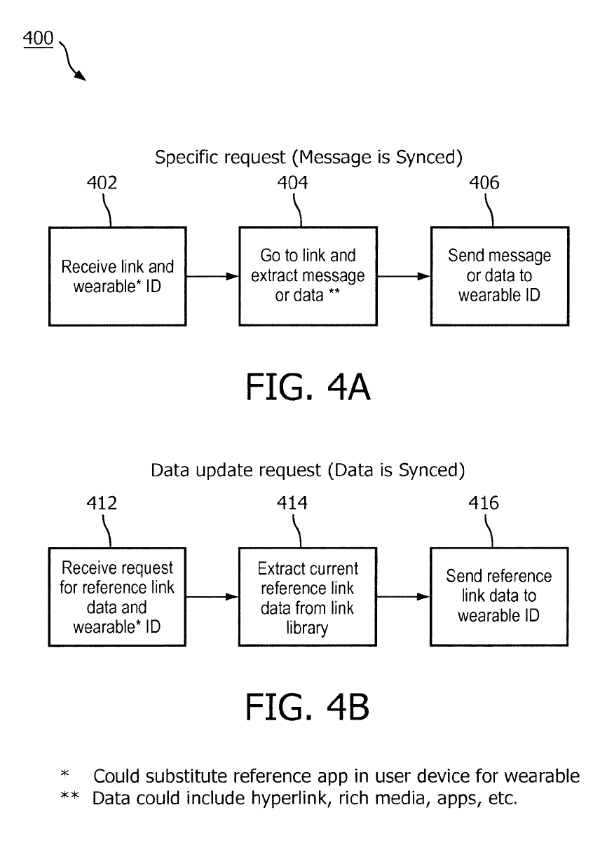

FIG. 4 illustrates an example of a method 400 for the link program as described herein. The link program may correspond, for example, to one or more of the link programs 136a,b,c described above with respect to FIG. 1. In particular, FIG. 4 shows two examples of operations the link program may undertake.

During a first operation (denoted as A), the link program may be used to address a particular request for information in the server. The request for information may pertain to a hyperlink request. In step 402, the reference instructions of the wearable device or the user device may have found an existing match in their respective reference link databases based on comparing received sensor data or calculated parameters to entries in the reference link database. The wearable device or the user device may then attempt to access the information from the hyperlink by sending an information request to the corresponding server associated with the hyperlink. The corresponding server may be included in the reference link database. The link program of the server receives the hyperlink from the user (e.g., wearable device or user device) as well as a corresponding identification of the user (e.g., wearable device identification, user device identification). For example a source IP or MAC address routinely included in various link requests may identify the user. Alternatively, the reference instructions or a third party platform between the user and servers (not shown) may be configured to append or otherwise include a user identifier in the request. The identification of the user may be used by the server to identify who is requesting the information and where the accessed information should be provided. In this step, a communication link may also be formed between the server and the user to facilitate in the transfer of information from the server to the user.

In step 404, the link program may access the hyperlink provided by the user and extract any information tied to that particular hyperlink. The information may include other hyperlinks, rich media, an application or anything that may be obtained from the cloud or Internet (e.g., documents, websites). Presumably the information obtained from the original hyperlink may be related to the sensor data obtained by the user.

After the data has been accessed, in step 406, the information is then provided to the user. By using the user identification, the server may determine where the information should be sent. The information may then be displayed on the wearable device or user device or stored in memory so that the user may access the information at a later time.

With reference to FIG. 4, the second operation of the link program is shown (denoted as B). In particular, the second operation pertains to the link program providing some or all available information in the link library of a server to update the reference link database wearable device or the user device.

In step 412, the link program may receive a request from the user to synchronize/update the reference link database in the wearable device/user device. The request to synchronize/update may also include the identification of the user where the information may be transmitted.

In step 414, the link program may extract some or all of the information stored in the link library. The request from the user in step 410 may be used to identify a particular subset of information that is stored in the link library that the user may be interested in. If that is the case, the link program may be instructed to only update the wearable device/user device with the selected information subset.

In step 416, the server may then provide the requested information to synchronize/update the wearable device/user device. The user identification may inform the server where the information should be sent.

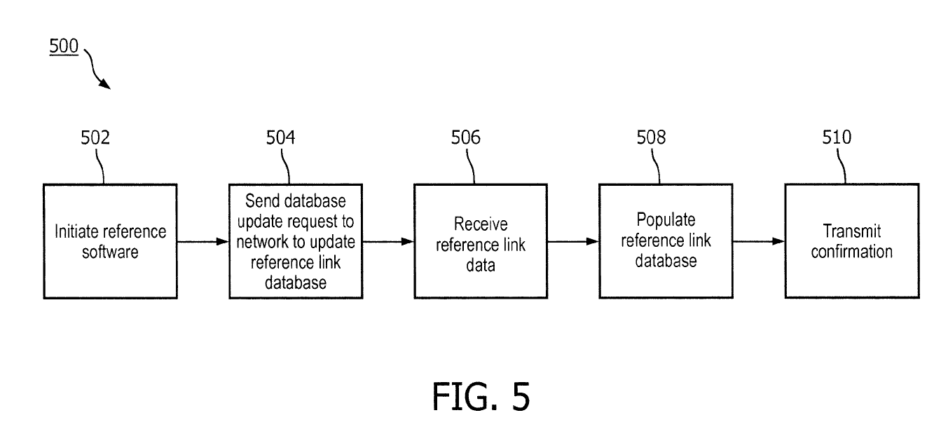

FIG. 5 illustrates an example of a method 500 for the reference instructions as described herein. In particular, the method 500 illustrates the steps taken by the reference instructions for updating/synchronizing the reference link database using information from the plurality of servers. It should be noted that the steps for the reference instructions may be performed by the reference instructions found in either the wearable device or the user device (see, FIG. 1).

In step 502, the reference instructions may be initiated. The reference instructions may be directly initiated by the user through user input. Alternatively, it may be possible that the reference instructions may be initiated to acquire information based on sensor data acquired. For example, if data being requested is not currently found in the reference link database, the reference instructions may look to the plurality of servers for the related information to update or synchronize the reference link database with.

In situations where related information cannot be found in the reference link database, the reference instructions may then, in step 504, provide an update request to a server to obtain information from the server to update or synchronize the reference link database in the wearable device. In some embodiments, the reference instructions may still request additional information from a server using an update request even if some information may already be stored in the reference link database initially. The request for additional information may be provided in order to ensure that the database is up-to-date and to make sure no additional information may be available. It should be noted that the update request being provided by the reference instructions may be the trigger for the link program to initiate the database update request method illustrated in FIG. 4 (see B). In this way, a communication channel may be formed between the user and the server to be used for any future transmission of data from the server to the user.

In step 506, once the information is found and accessed by the server, the reference instructions may then begin to receive the requested database information being transmitted from the server. The information being transmitted from the server may be transmitted using the server API.

In step 508, once the reference instructions receives all the requested information from the server, the reference instructions may then proceed to store the information into the reference link database. The reference link database may then be considered up-to-date or synchronized based on the available information (at the time) provided by the server. It should be noted that information in the server may constantly be updated by one or more parties using the server API. Therefore, in some embodiments, the reference instructions provide database update requests on a regular basis to ensure that the user possesses the most up-to-date information.

Once the population of the reference link database has been completed, the reference instructions, in step 510, may provide a confirmation to the server. The server provides this confirmation to the server to inform that the user reference link database has been updated. This confirmation may then allow the server to close communication.

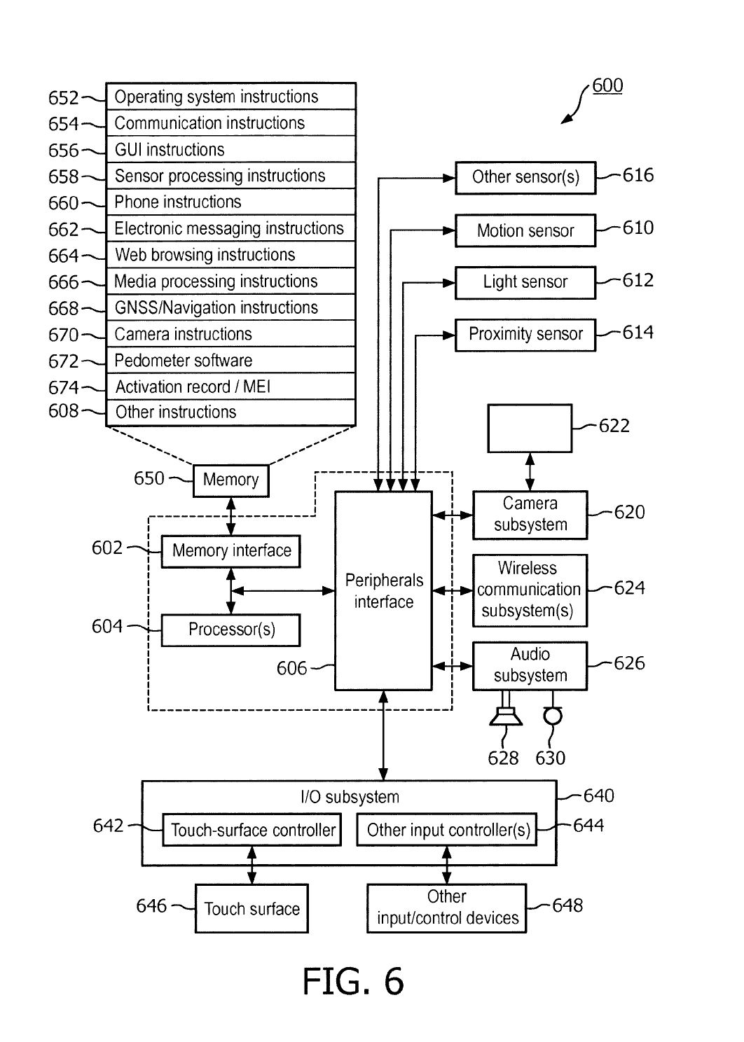

FIG. 6 illustrates an example of a computing device architecture 600 that may be utilized to implement the various features and processes described herein. For example, the computing device architecture 600 could be implemented in a pedometer. Architecture 600 as illustrated in FIG. 6 includes memory interface 602, processors 604, and peripheral interface 606. Memory interface 602, processors 604 and peripherals interface 606 may be separate components or may be integrated as a part of one or more integrated circuits. The various components may be coupled by one or more communication buses or signal lines.

Processors 604 as illustrated in FIG. 6 is meant to be inclusive of data processors, image processors, central processing unit, or any variety of multi-core processing devices. Additionally or alternatively, the processors 604 may include other logic devices such as FPGAs or ASICs. Any variety of sensors, external devices, and external subsystems may be in communication with the device 600 via peripherals interface 606 to facilitate any number of functionalities within the architecture 600 of the example mobile device. For example, motion sensor 610, light sensor 612, and proximity sensor 614 may be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be in communication with the peripherals interface 606, such as a temperature sensor, a biometric sensor, or other sensing device to facilitate corresponding functionalities. Location processor 615 (e.g., a global positioning transceiver) may be in communication with the peripherals interface 606 to allow for generation of geo-location data thereby facilitating geo-positioning. An electronic magnetometer 617 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor may facilitate camera functions such as recording photographs and video clips. Various additional or alternative sensors 616 may also be in communication with the peripherals interface 606.

Communication functionality may be facilitated through one or more communication subsystems 624, which may include one or more wireless communication subsystems. Wireless communication subsystems 624 may include 802.x or Bluetooth transceivers as well as optical transceivers such as infrared. Alternatively additionally, a wired communication system (no shown) may include a port device such as a Universal Serial Bus (USB) port or some other wired port connection that may be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 624 may include hosting protocols such that the device may be configured as a base station for other wireless devices. Communication subsystems may also allow the device to synchronize with a host device using one or more protocols such as TCP/IP, HTTP, or UDP.

Audio subsystem 626 may be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 may be in communication with a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 may display virtual or soft buttons and a virtual keyboard, which may be used as an input/output device by the user.

Other input controllers 644 may be in communication with other input/control devices 648 such as one or more buttons, rocker switches, thumb-wheels, infrared ports, USB ports, and/or a pointer device such as a stylus. The one or more buttons (not shown) may include an up/down button for volume control of speaker 628 and/or microphone 630. In some implementations, device 600 may include the functionality of an audio and/or video playback or recording device and may include a pin connector for tethering to other devices.

Memory interface 602 may be coupled to memory 650. Memory 650 may include high-speed random access memory or non-volatile memory such as magnetic disk storage devices, optical storage devices, or flash memory. Memory 650 may store operating system 652, such as Darwin, RTXC, LINUX, UNIX, OS X, ANDROID, WINDOWS, or an embedded operating system such as VxWorks. Operating system 652 may include instructions for handling basic system services and for performing hardware dependent tasks. In some implementations, operating system 652 may include a kernel.

Memory 650 may also store communication instructions 654 to facilitate communicating with other mobile computing devices or servers. Communication instructions 654 may also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; pedometer software 672, activation record/IMEI 674, and instructions 608 for any other application that may be operating on or in conjunction with the mobile computing device. [symbol] 650 may also store other software instructions for facilitating other processes, features and applications, such as applications related to navigation, social networking, location-based services or map displays.

Each of the above identified instructions and applications may correspond to a set of instructions for performing one or more functions described above. These instructions need not be implemented as separate software module programs, procedures, or modules. Memory 650 may include additional or fewer instructions. Furthermore, various functions of the mobile device may be implemented in hardware and/or in software module, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system may be connected by any form or medium of digital data communication such as a communication network. Some examples of communication networks include LAN, WAN and the computers and networks forming the Internet. The computer system may include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that may define on or more parameters that are passed between a calling application and other instructions such as an operating system, library routine, function that provides a service, that provides data, or that performs an operation or a computation. The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer may employ to access functions supporting the API. In some implementations, an API call may report to an application the capabilities of a device running the application, such as input capability, output capability, processing capability, power capability, and communications capability.

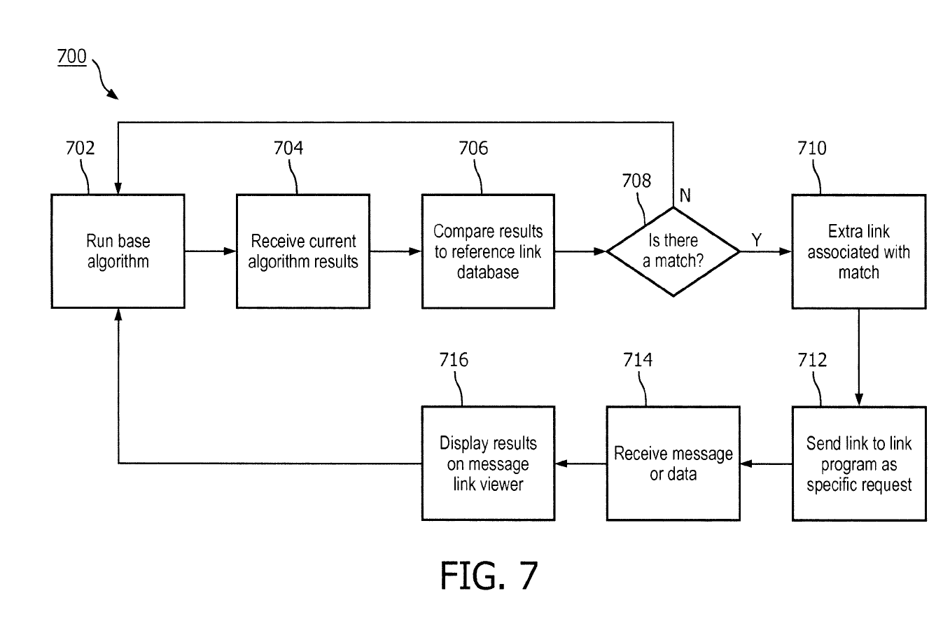

FIG. 7 illustrates an example of a method 700 whereby the reference instructions determine related hyperlinks as described herein. In particular, based on sensor data, parameters from the base algorithm instructions and/or one or more rules listed in the reference link database, the reference instructions may determine what information may be provided to the user.

In step 702, the base algorithm instructions may be initiated. The base algorithm instructions may use the sensor data obtained from the wearable device and calculate one or more parameters based on the sensor data. As indicated above, the sensors included in or otherwise in communication with the wearable device may obtain sensor data (e.g., steps taken) that may be used by the base algorithm instructions to calculate other parameters (e.g., calories burned, distance traveled). The sensor data and the parameters calculated from the base algorithm instructions may be provided to the reference software instructions to be used to determine the information the user may be interested. As noted above, the information may be stored in the reference link database. If the information is not currently found in the reference link database, a request may be sent out to update the database accordingly. In either case, the sensor data and the parameters may be used to determine what information the user may want to see.

In step 704, the reference instructions may receive the sensor data and/or the outputs from the base algorithm instructions. As noted above, the reference instructions may use the sensor data and/or the outputs from the base algorithm instructions to determine what types of information may be provided to the user.

In step 706, the reference instructions may use the sensor data and/or the output from the base algorithm instructions (e.g., parameters) to determine what information may be relevant to the user in the reference link database. As noted above, the entries in the reference link database may include classifications (e.g., data type, metric/limits) that may be used to specify which entry or entries in the reference link database may be applicable to the user. The reference instructions may use these classifications to narrow the available entries in the reference link database to one or more entries that most closely matches the sensor data and/or parameters provided. The matches may be indicative of the information that the user may be most interested in. It should be noted that in other embodiments, the reference instructions may be able to use other types of information (e.g., user input) to facilitate in determining what information may be relevant to the user.

In step 708, an output of step 706 is determined. In other words, the reference instructions may determine if there is any available information in the reference link database that may match the sensor data and/or parameters being used by the reference instructions. In a situation where there is no match available, the method up to this point as described in FIG. 7 may end. Afterwards, the reference instructions may re-start the method of FIG. 7 from the beginning by running the base algorithm instructions 702 again for new sensor data, if available. On the other hand, if there is a match found in step 708, the reference instructions may then proceed to the next step 710.

In step 710, the reference instructions may extract information from the reference link database from the one or more matched entries determined above in step 708. Generally the information extracted from the reference link database may include hyperlinks and servers. The hyperlinks may reference information available in the cloud or Internet related to the sensor data and/or parameters provided to the reference instructions. The servers identified in the reference link database may be servers that contain information related to the sensor data and/or parameters provided to the reference instructions.

In step 712, the reference instructions may use the information from the reference link database (e.g., hyperlink) to send appropriate information requests to the various servers over the cloud or Internet. For example, by using the server identification stored in the reference link database, the reference instructions may provide the hyperlink to the link program to the particular server. The server may then proceed to access and obtain the requested information. The information obtained from the hyperlink may be any type of information stored in the cloud or Internet that may be related to the sensor data and/or parameter used to determine the relevance of the hyperlink.

In step 714, the reference instructions receives the requested information obtained from the link transmitted to the server in step 712. The information obtained by the server may include a message or other types of data that the user may find useful based on the sensor data and/or parameters provided by the reference instructions. It should be noted that step 714 may be the step that initiates the link program method described above in FIG. 4 (see A).

In step 716, the reference instructions may display the information provided by the server on a display for the user to view. After the information has been displayed, the reference instructions may terminate and re-start back from the beginning with step 702 to use any new sensor data that may be available. It should be noted that the steps described above for the reference instructions may be performed in the wearable device and/or the user device.

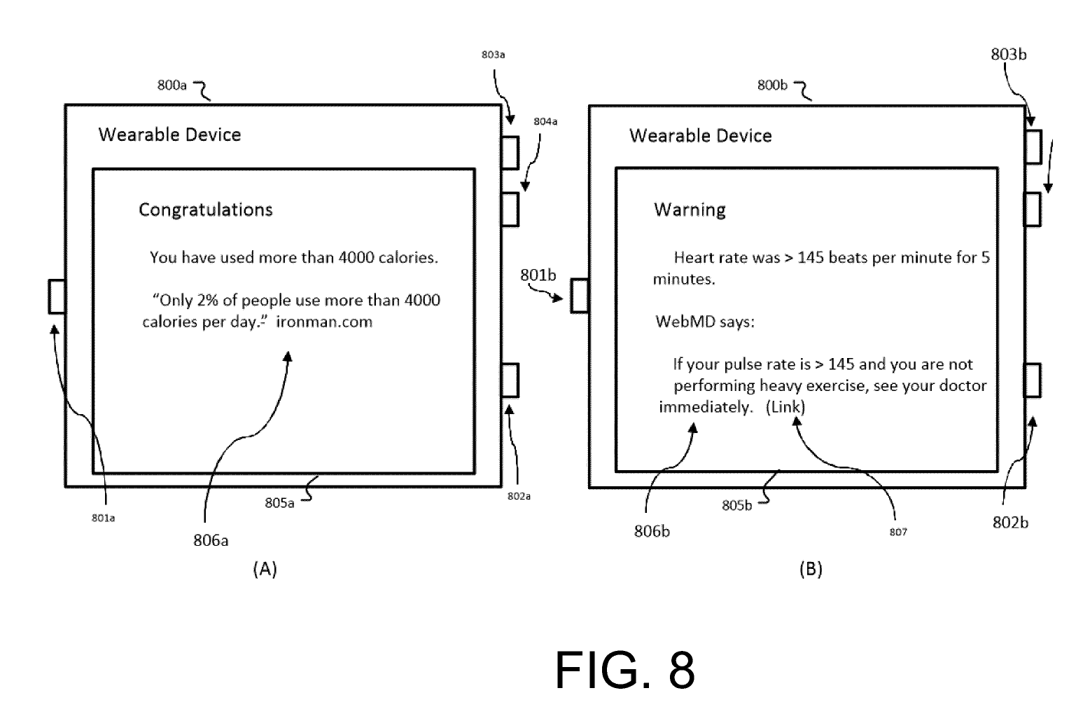

FIG. 8 illustrates an example of a display on the wearable device as described herein. In particular, the two example displays (see A and B) may be wearable devices 800a,b showing the two different types of information that may be displayed on the wearable device 800a,b for the user to view. The information may be the information received from servers based on available matches (see, for example, FIG. 7). In another embodiment, the information displayed on the display may be information directly retrieved from the reference link database. It should be noted that the displays in FIG. 8 may be any type of display in the art. In an embodiment of the present disclosure, the display may be a touch screen display.

With reference to the first display 805a (denoted as A) of the wearable device 800a, the associated wearable device 800a may include a plurality of buttons 801-804a. The plurality of buttons may be used, for example, as a way for the user to provide user input with the wearable device. As illustrated in FIG. 8, the buttons may allow the user to select one or more settings (e.g., settings button 801a), access other menu functions (e.g., menu button 802a) or provide the user a way to select and interact with various settings or features (e.g., up button 803a and down button 804a). The various settings and features may be displayed on the display 805a so that the user may use the up 803a and down buttons 804a to navigate the settings or features.

It should be noted that one or more of the buttons may also be replaced with other types of buttons, such as the crown button used in the APPLE WATCH device. Furthermore, additional buttons may be implemented in the wearable device 800a to facilitate additional functionality and interactivity between the user and the wearable device 800a.

As seen in the first display 805a, the display 805a may include a message 806a. The message 806a may be information that is extracted from the reference link database. The message 806a may also be information that is provided by the server based on requests sent by the reference instructions. At least in the particular embodiment illustrated in FIG. 8A, this message corresponds to the first entry found in the reference link database of FIG. 3. The example message provided recites “You have used more than 4000 calories. Only 2% of people use more than 4000 calories a day.” It should be noted that a variety of different types of messages may be displayed in a similar manner on the display 805a of the wearable device 800.

With reference to the second example wearable device 800b (denoted as B), the device includes a settings button 801b, menu button 802b, up button 803b, down button 804b, and a display 805b, the structure and functions of which will be apparent in view of the description of the first example wearable device 800a. The message 806b displayed on the wearable device 800b may also include a hyperlink 807. The hyperlink 807 may reference data corresponding to the user's health metrics or any other data which may be relevant to the message for the user to view. In an embodiment of the present disclosure, the user may be able to interact with the hyperlink using, for example the buttons or touch screen functionality of the display to instruct the wearable device to access information from the hyperlink. In such an embodiment, the data may be obtained in a similar manner as described above regarding the reference instructions (see, for example, FIG. 7).

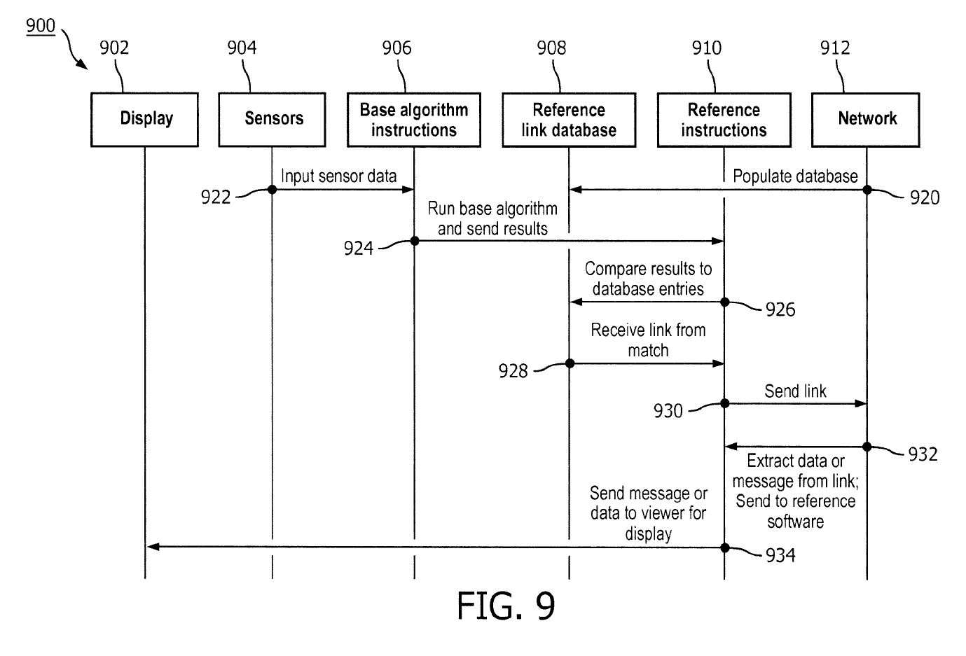

FIG. 9 illustrates an example swim lanes diagram among six elements (e.g., display 902, sensors 904, base algorithm instructions 906, reference link database 908, reference instructions 910, server 912, which may correspond to various components of the wearable device 110, user device 120, or other components of FIG. 1) of the system described herein.

In various embodiments, the swim lane illustration may be used to visually display the elements used in an overall method, the sequence of steps involved in the method and how the elements interact with each other. For example, an overall sequence of events in the process may be seen as the steps are shown from top to bottom (see steps 920-934). The horizontal divisions (e.g., the lines dictating the various elements) depict what element is performing that particular step (e.g., sensors, base algorithm instructions, reference link database). The arrows between the divisions represent how information or material is passed between elements (e.g., which elements are communicating with each other) (see steps 920-934). As illustrated in FIG. 9, the swim lane may show an overall sequence of processes of an example method.

In step 920, the server may populate information in the reference link database of the wearable device and/or the user device. This initial information may correspond to the currently available information from that particular server. In some situations, this information may be provided to the reference link database during a prior execution of the method shown by the diagram 900. Step 920 may correspond to a situation that the reference link database has some information stored that may be used by the wearable device and/or the user device.

In step 922, sensor data may be obtained from one or more sensors included in the wearable device. The sensor data may then be provided to the base algorithm instructions. As noted above, in FIG. 1, the base algorithm instructions may be used to calculate one or more outputs (e.g., parameters) using sensor data from the one or more sensors. The parameters may provide other types of information that may be shown to the user or be used to help the reference instructions determine what types of information the user may be interested in viewing.

In step 924, the base algorithm instructions may calculate the one or more outputs based on the sensor data received. As noted above, the outputs from the base algorithm instructions may provide additional information that may be used by the reference instructions or viewed by the user. The outputs are then provided to the reference software module to be used to determine what types of information may be of interest to the user.

In step 926, the reference instructions may determine what types of information may be of interest to the user by accessing the plurality of entries containing information (e.g., hyperlinks, server identification, messages) stored in the reference link database. The determination for what entries may be reached by comparing the output from the base algorithm instructions with one or more classifications for each of the entries within the reference link database. As illustrated in FIG. 3, the reference link database may include various types of information (e.g., data type, metric/limit) that may be used to classify a particular data entry in the reference link database. By looking and matching which entries may most relate to the outputs provided to the reference instructions, the reference instructions may obtain the stored information (e.g., hyperlinks, messages, server identification) that may most likely be of interest to the user.

In step 928, presuming there is at least one matching entry in the reference link database that most relates to the output from the base algorithm instructions being used by the reference instructions, the reference link database may transmit the requested information stored in the reference link database to the reference instructions. As noted above, the information may be hyperlinks, messages or a server identification where information may be found most relative to the output from the base algorithm instructions being used by the reference instructions. In situations where a hyperlink is received, the reference instructions may then perform steps to obtain the information associated with the received hyperlink.

In step 930, the reference instructions may begin a process to obtain the information associated with the received hyperlink from the reference link database. In particular, the reference instructions may provide the hyperlink to one or more servers in the cloud or Internet to see if a particular server has information tied to the hyperlink. In an embodiment of the present disclosure, the identity of the server or servers that have information on the hyperlink may be identified in the reference link database. In this embodiment, the reference instructions may directly transmit a data request related to the link to the particular server.

In step 932, a server that has been contacted by the reference instructions with a data request for information related to the transmitted hyperlink may then access the requested information for the reference instructions identified in the hyperlink. Once the server obtains the requested information (either from local storage or through requesting and receiving a remote resource via, e.g., an HTTP GET request), the information may then be transmitted back to the reference instructions.

In step 934, the reference instructions may display the information obtained from the hyperlink on a display (or message or link viewer) for the user to view. In an embodiment of the present disclosure, the reference instructions may also store the information in the reference link database or other memory locations in the wearable device and/or user device for future use. The information displayed on the display may include other hyperlinks, messages and data. The information being viewed by the user may be related to the sensor data and/or parameters calculated for which the wearable device is being used for. For example, in an embodiment of the present disclosure (see, for example FIG. 3), if sensor data and/or parameters are provided to the reference instructions relating to an amount of calories burned, a message that may be stored in the hyperlink of the reference link database, obtained from the server and eventually displayed on the display could be an inspirational message informing the user of exercise progress alongside personal statistics for the particular exercise session.

The reference instructions then provides the link to one or more servers (as seen in FIG. 1). The servers search and obtain, if available, related data or messages corresponding to the link provided by the reference instructions. Any data or messages obtained will then be provided back to the reference instructions. Finally, the reference instructions sends the message or data to the viewer so that the user may view.

As indicated above in FIG. 1, there may be a variety of functionalities for both the wearable device and the servers. The wearable devices may be used for a variety of different purposes, for example, monitoring biometrics of the user during exercise. Additionally, the servers may also be directed at various different uses. In FIG. 1, a server may be directed at medical monitoring to ensure, for example, that the user health is satisfactory. For some servers, there may be additional programs (e.g., emergency notification program) that may be included to facilitate the server.

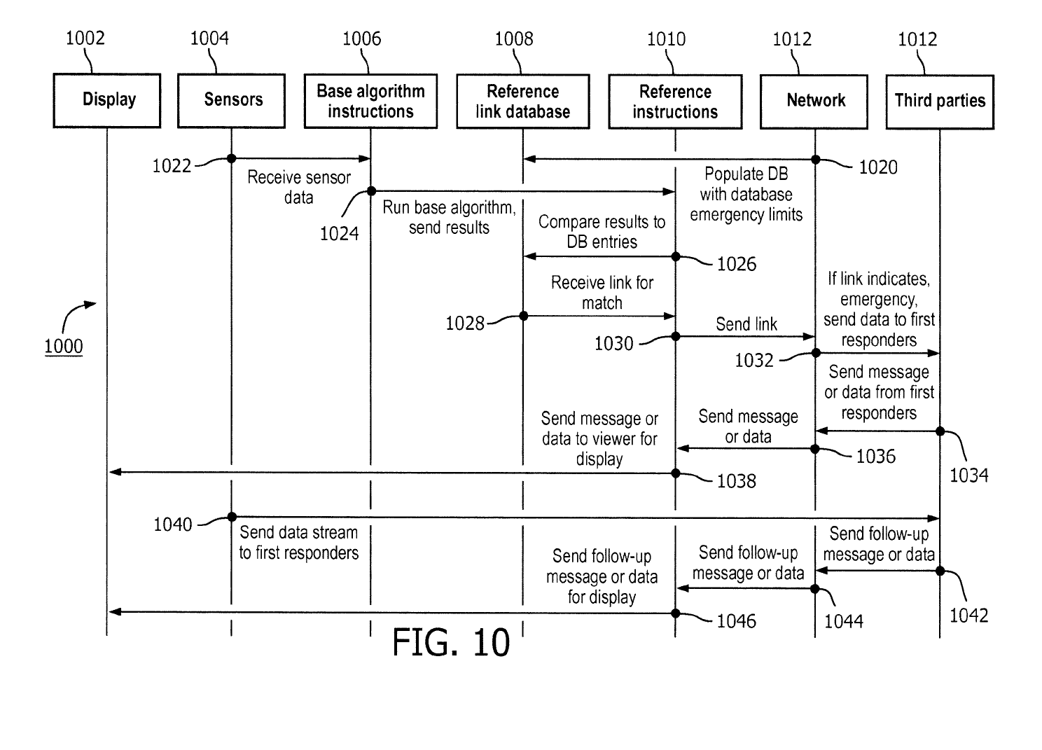

FIG. 10 illustrates an example of a swim lane diagram 1000 among seven elements (e.g., display 1002, sensors 1004, base algorithm instructions 1006, reference link database 1008, reference instructions 1010, server 1012, and some third party 1014) of the system as described herein. It should be noted that the swim lane diagram contains many of the similar steps illustrated above in FIG. 9. Based on the particular purpose of the medical monitoring server, however, additional steps may be included to carry out the functionality of the server.

In steps 1020, 1022, 1024, 1026, 1028, 1030, the process may initially be similar to the steps illustrated in FIG. 9. The steps relating to initial population of information in the link library 920, 1020 up until sending a data request regarding a hyperlink obtained from the reference link database to a particular server 930, 1030 may be replicated in the embodiment of FIG. 10 in a similar manner as in FIG. 9.

In step 1032, the server may evaluate the data request from the reference instructions associated with the hyperlink sent from the reference instructions to the server. In particular, based on the information extracted from the hyperlink, the medical monitoring server may provide related information to one or more other third parties. The information from the hyperlink may provide a determination whether sensor data and/or parameters raise, for example, an emergency condition. Third parties may be, for example, first responders, doctors, and other medical professionals. The third parties may be provided information based on the hyperlink if information associated with the hyperlink corresponds to an emergency condition. These third parties may also be provided information as a first step in assisting the user in the potential emergency condition.

In steps 1032, 1034, 1036, and 1038, the third parties (e.g., first responders) may then provide information to be eventually displayed on the display of the wearable device and/or user device for the user to view. The information may be transmitted in multiple steps (e.g., from the third party to the server to the user). In another embodiment, it may be possible that the information from the third party may be transmitted directly to the user. The information may include informing the user of a potential emergency condition arising. The information may also include further instructions to obtain more sensor data to confirm the possible emergency condition.

In step 1040, a data stream may be formed directly between the user and the third party (e.g., first responder). In particular the data stream may be used to provide sensor data directly to the first responders so that the first responders may monitor any sort of health metrics related to the possible emergency. Furthermore, this may allow the first responders to diagnose the user's condition en route to the location of the user. In some embodiments, the location of the user may be provided by the user. In other embodiments, the wearable device and/or the user device may include a global positioning system (GPS) receiver or other location aware device or service that may determine a user location and forward the user location to the first responder. In some embodiments, the user may be able to transmit messages back to the third party 1014 wither via the data stream 1040 or through sending a message in a manner similar, but reverse, to steps 1034, 1036, 1038, thereby achieving bidirectional communication with the third party.

In steps 1042, 1044, and 1046, the third party (e.g., first responder), may be able to provide one or more follow-up messages or data to the user. The follow-up messages, in some embodiments, may be provided through the medical emergency server and displayed for the user to view. In other embodiments, the third party may be able to provide follow-up messages directly to the user. These follow-up messages may be provided to obtain further information from the user (e.g., location, current perceived medical status). These follow-up messages may also be used to monitor the health condition of the user. For example, if the user is responsive, this may be indicative that the user is at least conscious.

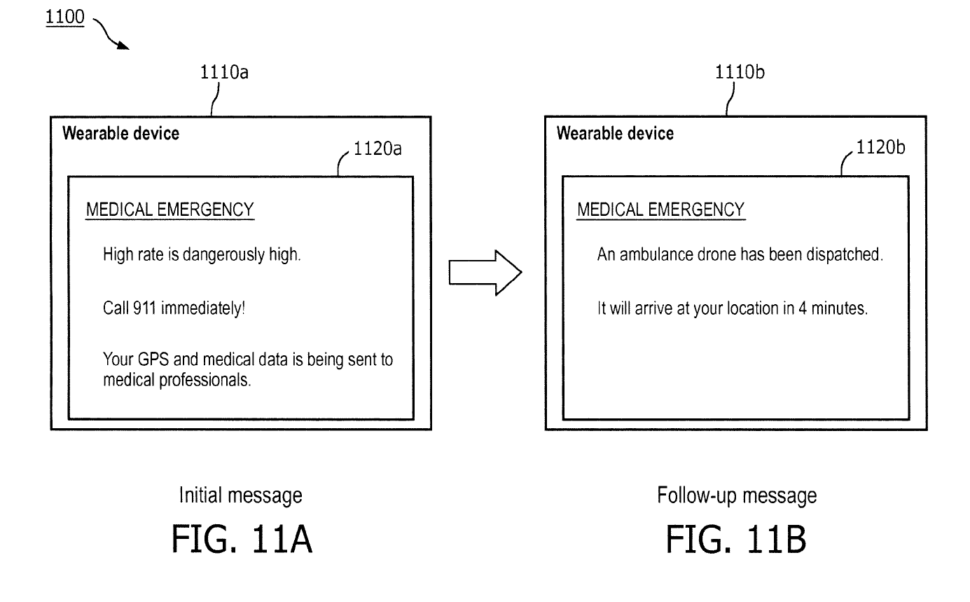

FIG. 11 illustrates examples of displays 1100 (denoted as A and B) used in conjunction with the medical emergency server as described herein. Similar to the embodiments illustrated in FIG. 8, a message related to a specific medical emergency may be provided on a display 1120a,b of a wearable device 1110a,b for the user to view. In particular, the display 1120a illustrates an examples of initial message provided from the third party to the user to view and B illustrates an example of a follow up message provided from the third party to the user. The example message on the display 1020a may be provided to the user to inform the user of the possible health condition that may be present based on sensor data and/or parameters calculated from the base algorithm instructions. The example message on the display 1020b may be provided, as a follow-up, to inform the user of medical assistance that is pending.

It should be apparent from the foregoing description that various exemplary embodiments of the invention may be implemented in hardware and/or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principals of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method of obtaining information based on a wearable device, the method comprising:
storing, in a memory associated with the wearable device, a record specifying applicability criteria, an internet resource identifier, and an internet server identifier;
obtaining, by a processor in communication with the memory, a user metric captured by the wearable device;
comparing the user metric to the applicability criteria to determine that the record is currently applicable;
transmitting the internet resource identifier to the internet server associated with the Internet server identifier;
receiving, in response to transmitting the internet resource identifier, data for display to a user wearing the wearable device; and
effecting display of the data to the user.

2. The method of claim 1, wherein
the memory and processor are components of a user device in communication with the wearable device;
obtaining the user metric comprises receiving the user metric from the wearable device; and
effecting display of the data to the user comprises displaying the data via a display device of the user device.

3. The method of claim 1, wherein
the memory and processor are components of a user device in communication with the wearable device; and
effecting display of the data to the user comprises transmitting the data to the wearable device.

4. The method of claim 1, wherein
the memory and processor are components of the wearable device, and
obtaining the user metric captured by the wearable device comprises reading the user metric from the memory, the method further comprising:
receiving, at the processor, first data from a sensor of the wearable device;
computing, by the processor second data based on the first data, wherein the user metric comprises at least one of the first data and the second data; and
storing the user metric in the memory.

5. The method of claim 1, wherein effecting display of the data to the user comprises displaying the data via a display device of the wearable device.

6. The method of claim 1, wherein the user metric is indicative of a medical emergency, the method further comprising:
after transmitting the internet resource identifier, receiving a message from a medical professional device associated with a medical professional; and
transmitting an additional user metric to the medical professional device, wherein the additional user metric comprises at least one of sensor data and computed data.

7. The method of claim 1, wherein the user metric is indicative of a medical emergency, the method further comprising, after transmitting the internet resource identifier, establishing a two-way communication session between the processor and a medical professional device associated with a medical professional.

8. The method of claim 1, further comprising:
receiving, from at least one of the server associated with the internet server identifier and an additional source server, at least one additional record specifying additional applicability criteria, an additional resource identifier, and at least one of: the server identified by the record, the additional source server, and an additional destination server; and
storing the additional record in the memory for future comparison to user metrics.

9. A wearable device for obtaining information, the device comprising:
a sensor configured to capture sensed data associated with a user wearing the wearable device;
a display device;
a communication interface;
a memory configured to store a record specifying applicability criteria, an internet resource identifier, and an internet server identifier; and
a processor configured to:
generate computed data based on the sensed data,
compare a user metric to the applicability criteria to determine whether the record is currently applicable, wherein the user metric comprises at least one of the sensed data and the computed data,
based on a determination that the record is currently applicable, transmit the internet resource identifier to the server associated with the internet server identifier via the communication interface,
receive, in response to transmitting the internet resource identifier and via the communication interface, response data for display to a user wearing the wearable device, and
control the display device to output a message to the user based on the response data.

10. The wearable device of claim 9, wherein the user metric is indicative of a medical emergency, the processor being further configured to:
after transmitting the internet resource identifier, receive a message from a medical professional device associated with a medical professional; and
transmit an additional user metric to the medical professional device, wherein the additional user metric comprises at least one of additional sensed data and additional computed data.

11. The wearable device of claim 9, wherein the user metric is indicative of a medical emergency, and the processor is further configured to, after transmitting the internet resource identifier, establishing a two-way communication session between the processor and a medical professional device associated with a medical professional.

12. The wearable device of claim 9, wherein the processor is further configured to
receive, from at least one of the server associated with the internet server identifier and an additional source server, at least one additional record specifying additional applicability criteria, an additional resource identifier, and at least one of: the server identified by the record, the additional source server, and an additional destination server; and
store the additional record in the memory for future comparison to user metrics.

13. A non-transitory machine-readable medium encoded with instructions for obtaining information based on a wearable device, the non-transitory machine-readable medium comprising:
instructions for storing, in a memory, a record specifying applicability criteria, an internet resource identifier, and an internet server identifier;
instructions for obtaining, by a processor in communication with the memory, a user metric captured by the wearable device;
instructions for comparing the user metric to the applicability criteria to determine that the record is currently applicable;
instructions for transmitting the internet resource identifier to the server associated with the internet server identifier;
instructions for receiving, in response to transmitting the resource identifier, data for display to a user wearing the wearable device; and
instructions for effecting display of the data to the user.

14. The non-transitory machine-readable medium of claim 13, wherein
the user metric is indicative of a medical emergency, the non-transitory machine-readable medium further comprising:
instructions for, after transmitting the internet resource identifier, receiving a message from a medical professional device associated with a medical professional; and
instructions for transmitting an additional user metric to the medical professional device, wherein the additional user metric comprises at least one of sensor data and computed data.

15. The non-transitory machine-readable medium of claim 13, further comprising:
instructions for receiving, from at least one of the server associated with the internet server identifier and an additional source server, at least one additional record specifying additional applicability criteria, an additional resource identifier, and at least one of: the server identified by the record, the additional source server, and an additional destination server; and
instructions for storing the additional record in the memory for future comparison to user metrics.
